(19) United States
(12) Patent Application Publication
(10) Pub. No.: US 2007/0260065 A1
(43) Pub. Date: Nov. 8, 2007

(54) PROCESS FOR PREPARING AMLODIPINE

(76) Inventors: Vijayabhaskar Bolugoddu, Hyderabad (IN); Jaydeepkumar Lilakar Dahyabhai, Valsad (IN); Ramachandra Reddy Pingili, Hyderabad (IN); Madhu Raju Veeraboina, Warangal (IN); Srinivas Reddy Gade, Hyderabad (IN); Srinivas Reddy Mallepalli, Hyderabad (IN); Ravindranath Tagore Amirisetty, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/741,801

(22) Filed: Apr. 30, 2007

Related U.S. Application Data
(60) Provisional application No. 60/807,628, filed on Jul. 18, 2006.

(30) Foreign Application Priority Data
May 3, 2006 (IN) 804/CHE/2006

(51) Int. Cl. C07D 213/06

(52) U.S. Cl. 546/252

(57) ABSTRACT

A process for preparing phthalimidoamlodipine, which is useful as an intermediate for the preparation of amlodipine and its salts.

### INTRODUCTION TO THE INVENTION

The present invention relates to a process for the preparation of amlodipine and its salts, and intermediates thereof. In particular it relates to process for the preparation of the compound phthalimidoamlodipine, which is useful as an intermediate for the preparation of amlodipine and its salts.

Amlodipine has a chemical name 3-Ethyl-5-methyl(±)-2-[(2-aminoethoxy)methyl]-4-(2-chlorophenyl)-1,4-dihydro-6-methyl-3,5-pyridinedicarboxylate and is structurally represented by Formula I.

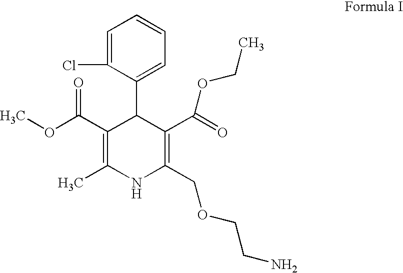

Amlodipine is useful for treating cardiovascular disease such as stenocardia, hypertension, and congestive cardioplegia. Pharmaceutical products containing amlodipine besylate are commercially available in the market as NORVASC™ in the form of white tablets equivalent to 2.5, 5, and 10 mg of amlodipine, to be administered orally.

European Patent No. 89,167 describes a class of dihydropyridine derivatives, one of them being amlodipine, and it also teaches the preparation of phthalimidoamlodipine and its conversion to amlodipine, and its pharmaceutically acceptable salts. Generally, the synthetic route disclosed in this patent for making amlodipine and other related dihydropyridine compounds comprises forming the corresponding amino-group protected precursor, followed by deprotection. Suitable protecting groups for the amino side chain group include benzylamino, dibenzylamino, azido, phthalimido groups. The precursor of amlodipine using the phthalimido protecting group is termed “phthalimidoamlodipine.”

Phthalimidoamlodipine has a chemical name 3-ethyl 5-methyl 2-[(2-phtalimidoethoxy)methyl]-4-(2-chlorophenyl)-1,4-dihydro-6-methyl-3,5-pyridine dicarboxylate and is represented by structural Formula II.

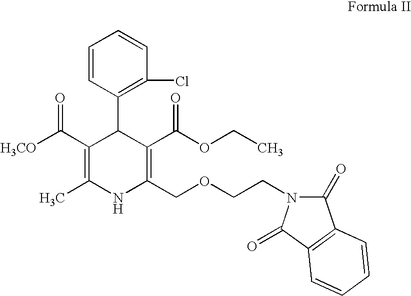

Phthalimidoamlodipine has certain advantages among other amino-protected precursors for amlodipine as it may be easily separated from the reaction mixture without danger (the azido-amlodipine is explosive) and is converted to amlodipine by simple, common deprotection procedures. It is thus considered to be a particularly useful key intermediate for the industrial production of amlodipine.

Various other processes for the preparation of phthalimido amlodipine have been described in International Application Publication Nos. WO 2002/53535, WO 2000/24714, in U.S. Pat. No. 6,653,481, and in Journal of Medicinal Chemistry, Vol. 29, 1986, pages 1696-1702.

International Application Publication No. WO 2006/003672 describes a process for the purification of phthalimido amlodipine in a combination of dichloromethane and n-hexane.

The processes described in the above documents suffer from drawbacks of either low purity or use of extensive work-up, and the reaction proceeds in two stages, which requires more processing time and using a large number of solvents.

None of the processes described in the above documents describe a one-pot synthesis for phthalimido amlodipine starting from [N-{2-hydroxyethyl}] phthalimide.

The present invention provides an optionally one-pot procedure for the preparation of phthalimidoamlodipine, and its conversion to amlodipine. The process of the present invention can be practiced on an industrial scale, and also can be carried out without sacrifice of overall yield based on the starting materials employed.

### SUMMARY OF THE INVENTION

The present invention relates to a process for the preparation of amlodipine and its salts, and intermediates thereof. In an aspect, it relates to a process for the preparation of phthalimidoamlodipine, which is useful as an intermediate for the preparation of amlodipine and its salts.

One aspect of the present invention provides a process for the preparation of phthalimidoamlodipine, comprising:

a) reacting [N-{2-hydroxyethyl}] phthalimide of Formula III with ethyl-4-chloro acetoacetate of Formula IV in the presence of a suitable base to afford ethyl 4-(2-(phthalimido) ethoxy) acetoacetate of Formula V, which optionally is not isolated;

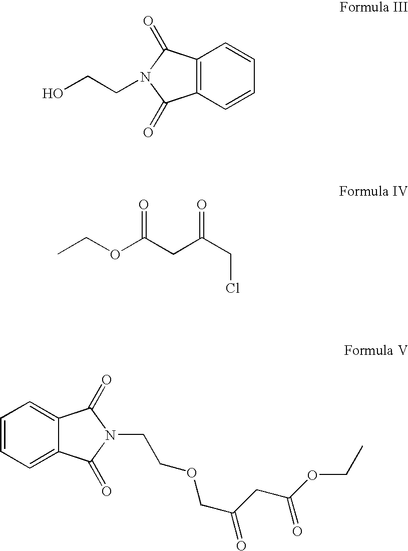

b) reacting 4-(2-(phthalimido) ethoxy) acetoacetate of Formula V with ortho chloro benzaldehyde of Formula VI in the presence of a suitable base and a halogenated solvent to afford 3-(2-chloro-phenyl)-2-{2-[2-(1,3-dioxo-1,3-dihydro-isoindol-2-yl)-ethoxy]-acetyl}-acrylic acid ethyl ester of Formula VII, which optionally is not isolated; and

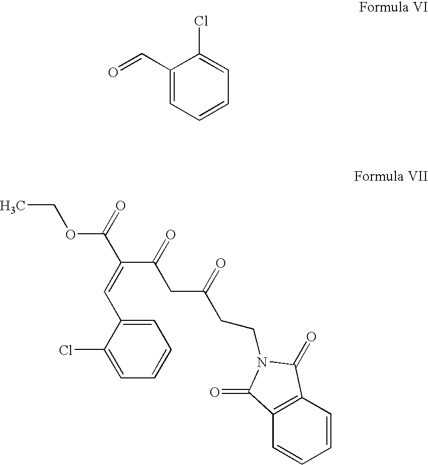

c) reacting 3-(2-Chloro-phenyl)-2-{2-[2-(1,3-dioxo-1,3-dihydro-isoindol-2-yl)-ethoxy]-acetyl}-acrylic acid ethyl ester of Formula VII with methyl aminocrotonate of Formula VII to afford phthalimidoamlodipine of Formula II.

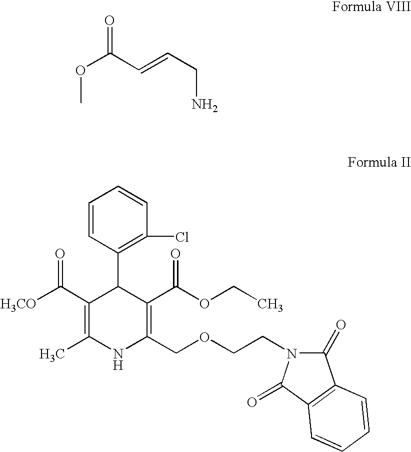

Phthalimidoamlodipine can be converted to amlodipine by reacting with aqueous methylamine, and amlodipine can be further converted to a pharmaceutically acceptable salt by reacting with the desired acid in a suitable solvent.

Another aspect of the present invention provides phthalimidoamlodipine, substantially free of a 4-(2-chloro phenyl)-2,6-dimethyl-1,4-dihydro pyridine-3,5-dicarboxylic acid dimethyl ester impurity of Formula IX

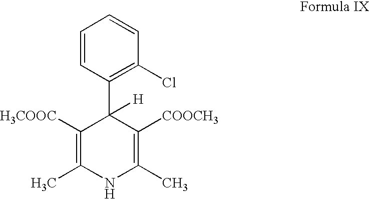

Still another aspect of the present invention provides a pharmaceutical composition comprising amlodipine or its pharmaceutically acceptable salt, prepared according to the process of the present invention, along with one or more pharmaceutically acceptable excipients.

An embodiment of the invention provides a process for preparing amlodipine or a salt thereof, comprising reacting a compound having a formula:

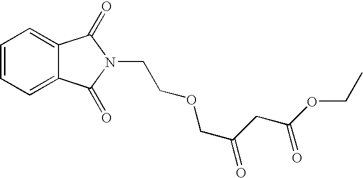

with ortho-chlorobenzaldehyde in the absence of a solvent or in the presence of a halogenated solvent to form a compound having a formula:

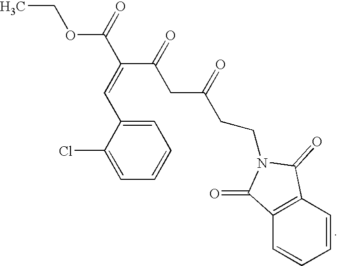

Another embodiment of the invention provides a compound having a formula:

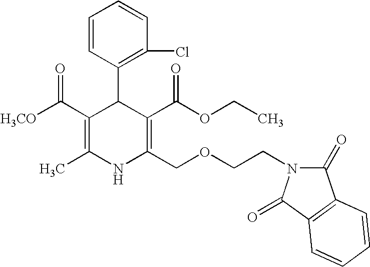

and containing less than about 1 percent by weight of a compound having a formula:

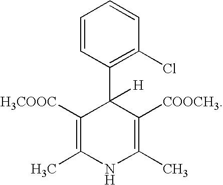

A further embodiment of the invention provides amlodipine or a salt thereof, containing less than about 0.5 percent by weight of a compound having a formula:

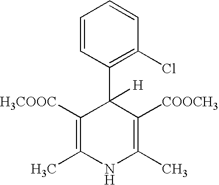

or a salt thereof.

### BRIEF DESCRIPTION OF THE DRAWING

FIG. 1 is a schematic representation of a process for preparing phthalimidoamlodipine.

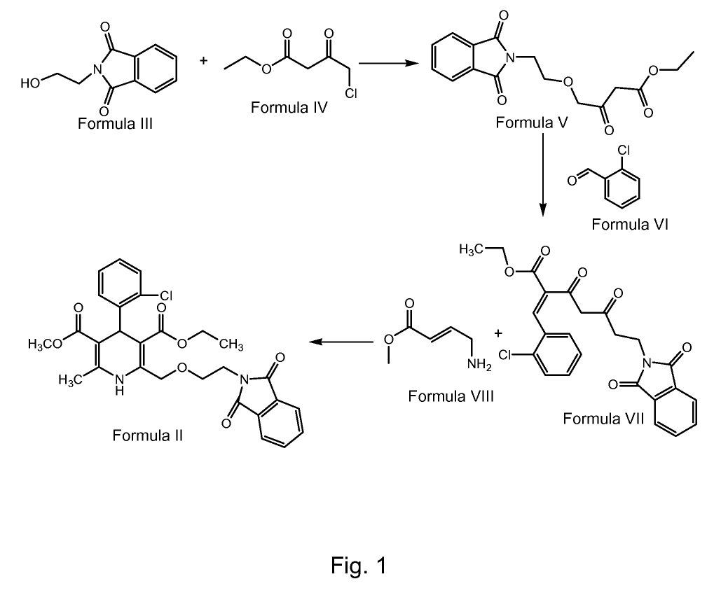

### DETAILED DESCRIPTION OF THE INVENTION

The present invention relates to a process for the preparation of amlodipine and its salts, and intermediates thereof. In an aspect, it relates to process for the preparation of phthalimidoamlodipine, which is useful as an intermediate for the preparation of amlodipine and its salts.

One aspect of the present invention provides a process for the preparation of phthalimidoamlodipine, comprising:

a) reacting [N-{2-hydroxyethyl}] phthalimide of Formula III with ethyl-4-chloro acetoacetate of Formula IV in the presence of a suitable base to afford ethyl 4-(2-(phthalimido) ethoxy) acetoacetate of Formula V, which optionally is not isolated;

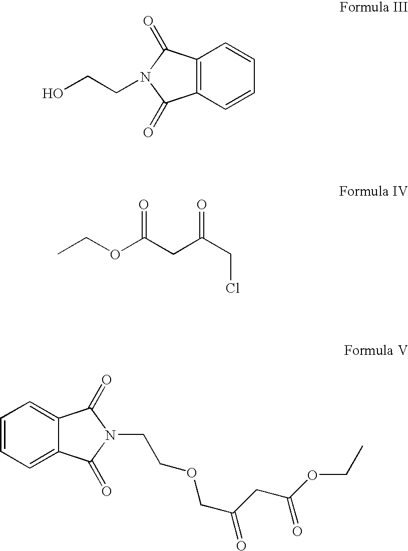

b) reacting 4-(2-(phthalimido) ethoxy) acetoacetate of Formula V with ortho-chlorobenzaldehyde of Formula VI in the presence of a suitable base and a halogenated solvent to afford 3-(2-chloro-phenyl)-2-{2-[2-(1,3-dioxo-1,3-dihydro-isoindol-2-yl)-ethoxy]-acetyl}-acrylic acid ethyl ester of Formula VII, which optionally is not isolated; and

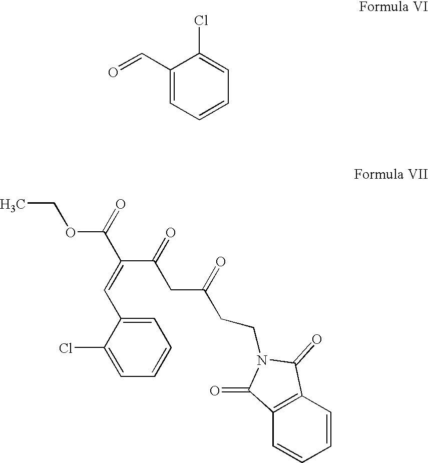

c) reacting 3-(2-chloro-phenyl)-2-{2-[2-(1,3-dioxo-1,3-dihydro-isoindol-2-yl)-ethoxy]-acetyl}-acrylic acid ethyl ester of Formula VII with methyl aminocrotonate of Formula VIII to afford phthalimido amlodipine of Formula II.

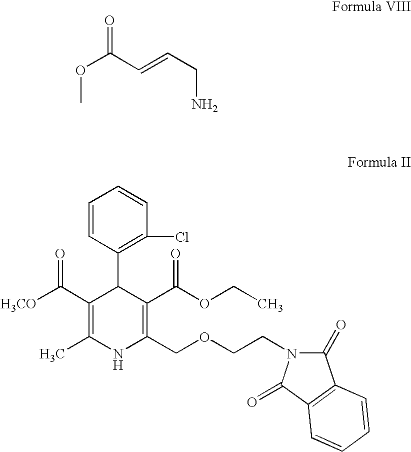

Step a) involves the reaction of [N-{2-hydroxyethyl}] phthalimide of Formula III with ethyl-4-chloroacetoacetate of Formula IV in the presence of a suitable base to afford ethyl 4-(2-(phthalimido) ethoxy) acetoacetate of Formula V, which optionally is not isolated.

Suitable solvents which can be used for the reaction include, but are not limited to: hydrocarbons such as toluene, xylene, n-heptane, cyclohexane, n-hexane and the like; nitriles such as acetonitrile, propionitrile and the like; halogenated hydrocarbons such as dichloromethane, 1,2-dichloroethane, chloroform, carbon tetrachloride and the like; and mixtures thereof.

Suitable bases which can be used include, but are not limited to: alkali metal hydrides such as lithium hydride, sodium hydride and the like; alkali metal hydroxides such as lithium hydroxide, sodium hydroxide, potassium hydroxide and the like; carbonates of alkali metals such as sodium carbonate, potassium carbonate and the like; bicarbonates of alkali metals such as sodium bicarbonate, potassium bicarbonate, and the like; ammonia; and mixtures thereof. These bases can be used in the form of solids or in the form of aqueous solutions.

Suitable temperatures for conducting the reaction range from about 0 to about 100° C., or from about 10 to about 70° C.

In an embodiment, [N-{2-hydroxyethyl}] phthalimide is added to a mixture of the base and the organic solvent in small portions followed by the addition of ethyl-4-chloroacetoacetate. The addition of [N-{2-hydroxyethyl}] phthalimide in small portions aids in uniform contact of the reactants with the reagents resulting in quick completion of the reaction, and thus increasing the yields of the product.

The ethyl 4-(2-(phthalimido) ethoxy) acetoacetate of Formula V, formed in the reaction mixture, optionally is not isolated and is directly progressed to step b).

Step b) involves reaction of 4-(2-(phthalimido) ethoxy) acetoacetate of Formula V with ortho-chlorobenzaldehyde of Formula VI in the presence of a suitable base and a halogenated solvent to afford 3-(2-chloro-phenyl)-2-{2-[2-(1,3-dioxo-1,3-dihydro-isoindol-2-yl)-ethoxy]-acetyl}-acrylic acid ethyl ester of Formula VII, which optionally is not isolated prior to further reaction.

Suitable halogenated solvents which can be used for conducting the reaction include, but are not limited to, halogenated hydrocarbons such as dichloromethane, 1,2-dichloroethane, chloroform, carbon tetrachloride and the like, and mixtures thereof.

Suitable bases which can be used for the reaction include, but are not limited to: organic bases such as pyridine, triethylamine, dimethylamine, methylamine, aqueous ammonia, and the like; and inorganic bases such as sodium hydroxide, potassium hydroxide, sodium carbonate, potassium carbonate, sodium bicarbonate, potassium bicarbonate, and the like.

Suitable temperatures for conducting the reaction range from about 10° C. to about 100° C., or from about 20° C. to about 70° C.

The reaction is moisture sensitive, hence the individual components used in the reaction like solvents, bases, and reagents should be used in an anhydrous condition and moisture content should be less than or equal to 0.1% w/v. If the moisture content is more than the 0.2% w/v, the reaction may not proceed efficiently and may result in the formation of side products.

In an embodiment, the reaction is conducted in the presence of molecular sieves. Molecular sieves are used to remove water from the reaction mixture. Three and four angstrom molecular sieves are examples of those that can be used, although other molecular sieves are also useful. The molecular sieves may be mixed with the reaction mixture to remove water from the reaction mixture.

A solvent or reagent substantially free of water may also be obtained by drying the solvent with molecular sieves, or any other drying agents like sodium sulfate, magnesium sulfate, or the like,

In an embodiment, the reaction is conducted in the absence of an organic solvent.

The 3-(2-chloro-phenyl)-2-{2-[2-(1,3-dioxo-1,3-dihydro-isoindol-2-yl)-ethoxy]-acetyl}-acrylic acid ethyl ester of Formula VII, formed in the reaction mass, is present in an organic layer and optionally is not isolated, but progressed directly to the next stage.

Step c) involves the reaction of 3-(2-chloro-phenyl)-2-{2-[2-(1,3-dioxo-1,3-dihydro-isoindol-2-yl)-ethoxy]-acetyl}-acrylic acid ethyl ester of Formula VII with methyl aminocrotonate of Formula VIII to afford phthalimidoamlodipine of Formula II.

Suitable solvents which can be used for the reaction include, but are not limited to: hydrocarbons such as toluene, xylene, n-heptane, cyclohexane, n-hexane and the like; nitriles such as acetonitrile, propionitrile and the like; halogenated hydrocarbons such as dichloromethane, 1,2-dichloroethane, chloroform, carbon tetrachloride and the like; and mixtures thereof.

Suitably, the reaction is conducted under acidic conditions. The acidic conditions can be provided using acids such as but not limited to: inorganic acids such as hydrochloric acid, hydrobromic acid, and the like; and organic acids such as oxalic acid, trifluoroacetic acid, tartaric acid, formic acid, acetic acid, para-toluene sulfonic acid and the like.

Suitable temperatures for conducting the reaction range from about 0 to about 100° C., or from about 20 to about 80° C.

Pthalimidoamlodipine obtained above can be optionally purified further by recrystallization or slurrying in a suitable solvent.

Suitable solvents which can be used include, but are not limited to: ethyl acetate, n-propyl acetate, n-butyl acetate, t-butyl acetate; halogenated hydrocarbons such as dichloromethane, ethylene dichloride, chloroform, and the like; nitriles such as acetonitrile, propionitrile, and the like; and mixtures thereof.

For recrystallization, a solution can be prepared at an elevated temperature, if desired, to achieve a desired high solute concentration. Any temperature is acceptable for the dissolution as long as a clear solution of the phthalimidoamlodipine is obtained and is not detrimental to the drug substance chemically or physically. The solution may be brought down to lower temperatures, such as in the range of −5 to 30° C., for further isolation if required or an elevated temperature may be used.

The purified compound may be further dried before proceeding to the next stage. The drying can be carried out at temperatures of about 35° C. to about 70° C. The drying can be carried out for any desired time periods to obtain a desired product purity, such as from about 1 to 20 hours, or longer.

Phthalimidoamlodipine can be converted to amlodipine by reacting with aqueous methylamine, and the amlodipine can be further converted to a pharmaceutically acceptable salt by reacting with the desired acid in a suitable solvent.

Another aspect of the present invention provides phthalimidoamlodipine, substantially free of a 4-(2-chloro phenyl)-2,6-dimethyl-1,4-dihydropyridine-3,5-dicarboxylic acid dimethyl ester impurity of Formula IX.

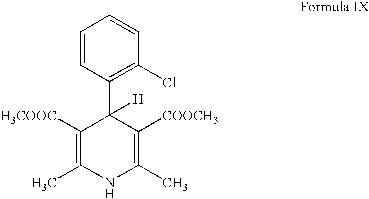

Phthalimidoamlodipine prepared according to the process of the present invention typically contains less than about 1%, or less than about 0.5%, by weight of 4-(2-chlorophenyl)-2,6-dimethyl-1,4-dihydro pyridine-3,5-dicarboxylic acid dimethyl ester of Formula IX as an impurity. Subsequently, amlodipine or a salt thereof prepared from the phthalimidoamlodipine also is substantially free of the impurity, or a salt thereof, and frequently contains less than about 0.5%, or less than about 0.15%, by weight of the impurity or its salt.

Still another aspect of the present invention provides a pharmaceutical composition comprising amlodipine or its pharmaceutically acceptable salt, prepared according to the process of the present invention, together with one or more pharmaceutically acceptable excipients.

The pharmaceutical composition comprising amlodipine or its salts may be further formulated into solid oral dosage forms such as, but not limited to, powders, granules, pellets, tablets, and capsules; liquid oral dosage forms, such as but not limited to, syrups, suspensions, dispersions, and emulsions; and injectable preparations, such as but not limited to, solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared by direct blending, dry granulation or wet granulation or by extrusion and spheronization. Compositions may be presented as uncoated, film coated, sugar coated, powder coated, enteric coated or modified release coated. Compositions of the present invention may further comprise one or more pharmaceutically acceptable excipients.

Pharmaceutically acceptable excipients that find use in the present invention include, but are not limited to: diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinyl pyrrolidone, hydroxypropyl cellulose, hydroxypropyl methylcellulose, pregelatinized starch and the like; disintegrants such as starch, sodium starch glycolate, pregelatinized starch, crospovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins, resins; release rate controlling agents such as hydroxypropyl cellulose, hydroxymethyl cellulose, hydroxypropyl methylcellulose, ethyl cellulose, methyl cellulose, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants and the like.

In the compositions of the present invention, amlodipine is a useful active ingredient in the range of about 1 to about 10 mg, per dosage unit.

Certain specific aspects and embodiments of this invention are described in further detail by the examples below, which examples are provided only for the purpose of illustration and are not intended to limit the scope of the invention as defined by the appended claims in any manner.

### EXAMPLE 1

### Preparation of Ethyl 4-(2-(Phthalimido)Ethoxy)Acetoacetate (Formula V)

300 liters of toluene was taken into a clean and dry reactor and 50 Kg of sodium hydride was added under a nitrogen atmosphere. 50 Kg of [N-{2-hydroxyethyl}] phthalimide was added to the reactor and the resultant reaction mass was subjected to agitation and heating to 57° C. The reaction mass was maintained at 56-57° C. for 30 minutes. Another 50 Kg of [N-{2-hydroxyethyl}] phthalimide was added to the above reaction mass in 18 lots of 2.7 kg each and a further lot of 1.4 kg, at 60° C. Liberation of hydrogen was observed. The reaction mass was maintained at 57° C. until hydrogen gas liberation ceased. A solution of 94 Kg of ethyl 4-chloroacetoacetate in 287 liters of toluene was added to the above reaction mass at 57° C. and maintained at the same temperature for 20 minutes. The reaction mass was then cooled to 30° C. and then further cooled to 17° C. 200 liters of glacial acetic acid was added to the above obtained reaction mass in three equal lots at 17° C. and stirred for 30 minutes. 1000 liters of water was added to the reaction mass and stirred for 20 minutes. The organic layer was separated and the aqueous layer was extracted into 220 liters of toluene. The combined toluene layer was progressed to the next stage.

### EXAMPLE 2

### Preparation of Phthalimidoamlodipine (Formula II)

750 liters of the organic layer obtained by a process similar to Example 1 was taken into a clean and dry reactor and subjected to distillation at a temperature of 52° C., under a vacuum of 650 mm Hg. The residue obtained was cooled to 30° C. and 750 liters of dichloromethane was added to it and stirred for 45 minutes. 4.6 kg of piperidine and 2.4 liters of glacial acetic acid was added to the reaction mass and stirred for 30 minutes at 32° C. 52.5 kg of ortho-chlorobenzaldehyde was then added and the reaction mass was heated to 39° C. The reaction mass was maintained at 39° C. for 12.5 hours. Reaction completion was checked using thin layer chromatography. After the reaction was complete, the reaction mass was cooled to 27° C. and 300 liters of water was added and stirred for 15 minutes. The organic layer was separated and cooled to 13° C. and 565 liters of glacial acetic acid was added at the same temperature. After the addition was complete, the solvent was distilled completely at a temperature of 40 to 43° C. under a vacuum of 420 mm Hg. Another 75 liters of glacial acetic acid was added to the obtained residue and cooled to 15° C. 762.4 liters of methyl 3-aminocrotonate was added to the reaction mass at 12 to 15° C. After completion of addition, the temperature of the reaction mass was raised to 28° C. and maintained for 23 hours. The reaction mass was then filtered and the filtered solid was washed with 120 liters of dichloromethane. The wet material was spin dried for 1.5 hours. The semi-wet material was taken into another reactor and 150 liters of ethyl acetate was added. The reaction mass was stirred for 10 minutes and then heated to 76° C. to form a clear solution. After a clear solution was obtained, the solution was cooled to 30° C. and then further cooled to 2 to 3° C. and maintained for 1 hour. The formed solid was filtered and washed with 50 liters of ethyl acetate. The wet material was dried in a spin drier for 3 hours and then further dried in an air drier at 67° C. for 5 hours to yield 82.5 kg of the title compound.

Purity by HPLC: 99.3% by weight.

### EXAMPLE 3

### Preparation of Phthalimidoamlodipine (Formula II)

200 g of ethyl 4-(2-(phthalimido) ethoxy) acetoacetate, obtained by disitillation of the organic layer obtained in Example 1, and 2000 ml of dichloromethane were taken into a round bottom flask and 11.6 g of piperidine was added, followed by addition of 64 ml of glacial acetic acid and 44 g of ortho-chlorobenzaldehyde. The reaction mass was stirred at a temperature of 40° C. for 5 hours. Another 88 g of the o-chlorobenzaldehyde was added to the reaction mass in three equal lots and stirred at 40° C. for 30 minutes. The reaction mass was then distilled completely in a Buchi Rotavapor at a temperature of 40° C., and the obtained residue was cooled to 25° C. 1500 ml of glacial acetic acid was added to the residue and then 240 g of methyl-3-aminocrotonate was added. The reaction mass was stirred at 30° C. for 20 hours. The reaction mass was then filtered and the wet solid was taken into another round bottom flask and 300 ml of ethyl acetate was added to it. The mixture was heated to 78° C. to obtain a clear solution. The solution was cooled to 5° C. and maintained for 40 minutes. The formed solid was filtered and washed with 100 ml of ethyl acetate. The wet solid was dried at 60° C. for 2 hours to yield 259 g of the title compound.

### EXAMPLE 4

### Preparation of Phthalimidoamlodipine in the Presence of Molecular Sieves

130 g of 4-(2-(phthalimido) ethoxy) acetoacetate obtained using a processs similar to the one described in Example 1 and 500 ml of dichloromethane were taken into a round bottom flask and 4.3 g of piperidine was added. 2.4 ml of glacial acetic acid, 24.8 g of o-chlorobenzaldehyde, and 35 g of molecular sieves (4A, 1.5 mm pellets, Loba Chemie Pvt. Ltd., Mumbia, India) were then added under stirring. The reaction mass was heated to a temperature of 40° C. and maintained for 14 hours. Reaction progress was monitored using thin layer chromatography. After the reaction was complete, the reaction mass was filtered and the solid was washed with 100 ml of dichloromethane. The filtrate was distilled completely at a temperature of 40° C. To the resulting residue, 562 ml of glacial acetic acid and 90.3 g of methyl 3-aminocrotonate were added and stirred at 28° C. for 15 hours. The reaction mass was then filtered and the wet compound was taken into 112.5 ml of ethyl acetate. The mixture was heated to 78° C. to form a clear solution. The solution was cooled to 5° C. and maintained for 40 minutes. The formed solid was filtered and washed with 10 ml of ethyl acetate. The wet solid was dried at 60° C. for 2 hours to yield 103.3 g of the title compound.

### EXAMPLE 5

### Preparation of Phthalimidoamlodipine in the Absence of Solvents

50 g of 4-(2-(phthalimido) ethoxy) acetoacetate obtained by a process similar to Example 1 and 19.8 g of o-chlorobenzaldehyde were taken into a round bottom flask and stirred, followed by the addition of 0.23 g of piperidine. 0.16 ml of glacial acetic acid was added to the reaction mass under a nitrogen atmosphere and the mixture was heated to a temperature of 60° C. and maintained at 60° C. for 4.5 hours. Then 200 ml of glacial acetic acid was added followed by the addition of 34.2 g of methyl 3-aminocrotonate. The reaction mass was maintained at 25 to 27° C. for 24 hours. The reaction mass was then filtered and the solid was washed with 10 ml of acetic acid. The wet solid was taken into a fresh round bottom flask and 60 ml of ethyl acetate was added and heated to 75° C. to form a clear solution. The solution was cooled to 5° C. and maintained for 40 minutes. The formed solid was filtered and washed with 15 ml of ethyl acetate. The wet solid was dried at 60° C. for 2 hours to yield 39.4 g of the title compound.

### EXAMPLE 6

### Preparation of Amlodipine Free Base from Phthalidoamlodipine

70 kg of phthalimido amlodipine obtained above and 420 liters of methylamine solution (40.66% w/v) were taken into a reactor and stirred at 30° C. for 25 hours. The reaction mass was then filtered and the filter cake was washed with 140 liters of water. The wet material was suction dried under a vacuum of 650 mm Hg for 2 hours. The wet material was again taken into another reactor, 210 liters of water was added and the mixture was stirred for 1 hour, 10 minutes at 28° C. The solid was filtered and washed with 35 liters of water and suction dried under vacuum for 1 hour. The wet material was again taken into another reactor and 350 liters of water was added. 9 liters of glacial acetic acid was added to the reactor and stirred for 10 minutes. The reaction mass was checked for formation of a clear solution and then filtered. The filtrate was taken into another reactor and a solution of 140 liters of water and 0.5 liters of glacial acetic acid were added. Then 145 liters of methanol was added, and pH of the mixture was adjusted to 10.5 with 40% aqueous methylamine solution. The reaction mass was stirred for 30 minutes and filtered. The solid was washed with 140 liters of water. The wet material was dried at 61° C. for 10 hours to yield 39 kg of the title compound.

Purity by HPLC: 99.06% by weight.

### EXAMPLE 7

### Preparation of Amlodipine Besylate from Amlodipine Free Base

150 liters of isopropyl alcohol and 12 kg of benzene sulfonic acid were taken into a reactor and heated to 72° C. to form a clear solution. 120 liters of isopropyl alcohol was added followed by addition of 30 kg of amlodipine free base at 43° C. The reaction mass was stirred for 30 minutes at 43° C. and then cooled to 27° C., and again stirred for 30 minutes. The formed solid was filtered and washed with 25 liters of isopropyl alcohol and spin dried for 1 hour. The wet material was taken into another reactor and 135 liters of isopropyl alcohol was added. The mixture was heated to 72° C., 8 liters of water was added, and the reaction mass was stirred at 72° C. for 15 minutes to form a clear solution, then another 15 liters of isopropyl alcohol was added. 90 liters of petroleum ether was then added and the mass was cooled to 27° C., then further cooled to 2° C. and maintained for 1 hour. The formed solid was filtered and washed with 15 liters of isopropyl alcohol. The wet material was spin dried for 4 hours and then dried at a temperature of 67° C. and a vacuum of 600 mm Hg for 3 hours to yield 30.8 kg of the title compound.

Purity by HPLC: 99.2% by weight.

Residual solvent content: methanol: Less than 10 ppm, isopropyl alcohol: 576 ppm, petroleum ether: 171 ppm.

Particle size distribution: D₁₀ less than 6 μm; D₅₀ less than 16 μm; D₉₀ less than 35 μm.

Moisture content: 0.03 by weight.

We claim:

1. A process for preparing amlodipine or a salt thereof, comprising reacting a compound having a formula: 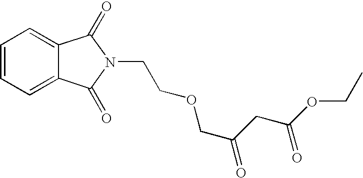
with ortho-chlorobenzaldehyde in the absence of a solvent or in the presence of a halogenated solvent to form a compound having a formula:

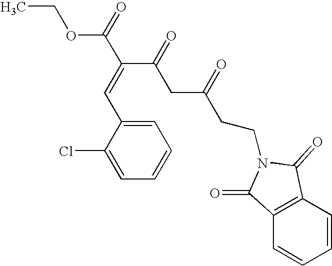

2. The process of claim 1, wherein a reaction with ortho-chlorobenzaldehyde is conducted in the absence of a solvent.

3. The process of claim 1, wherein a reaction with ortho-chlorobenzaldehyde is conducted in the presence of a halogenated solvent.

4. The process of claim 1, wherein a reaction with ortho-chlorobenzaldehyde is conducted in the presence of a halogenated solvent comprising dichloromethane.

5. The process of claim 1, which is carried out in the presence of a moisture absorbent.

6. The process of claim 5, wherein a moisture absorbent comprises molecular sieves.

7. The process of claim 1, wherein a product from reacting with ortho-chlorobenzaldehyde is not isolated before further reaction.

8. The process of claim 1, further comprising reacting a compound having a formula: 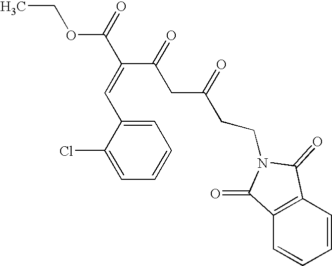
with methyl 3-aminocrotonate to form a compound having a formula:

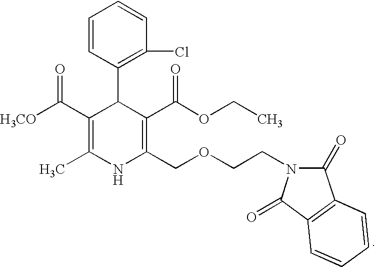

9. The process of claim 8 further comprising reacting a compound having a formula: 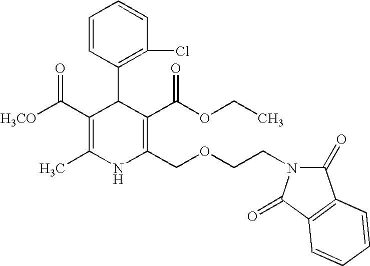
with aqueous methylamine to form amlodipine.

10. The process of claim 1, wherein a compound having a formula: 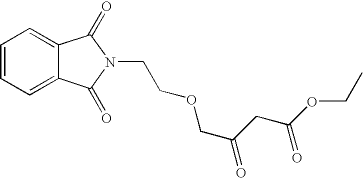
is prepared by reacting a compound having a formula: 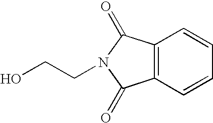
with ethyl 4-chloroacetoacetate.

11. The process of claim 10, wherein a product of reacting with ethyl 4-chloroacetoacetate is not isolated before further reaction.

12. The process of claim 1, wherein a product from reacting with ortho-chlorobenzaldehyde is not isolated before further reaction, and a product of reacting with ethyl 4-chloroacetoacetate is not isolated before further reaction.

13. A compound having a formula: 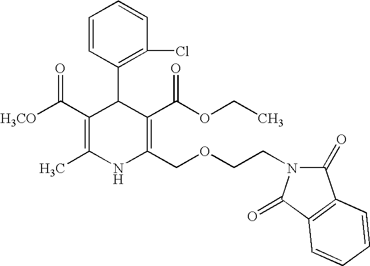
and containing less than about 1 percent by weight of a compound having a formula:

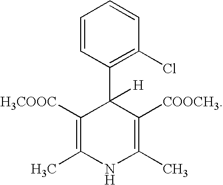

14. The compound of claim 13, containing less than about 0.5 percent by weight of a compound having a formula:

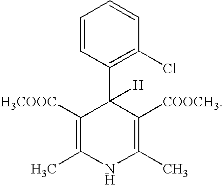

15. Amlodipine or a salt thereof, containing less than about 0.5 percent by weight of a compound having a formula: 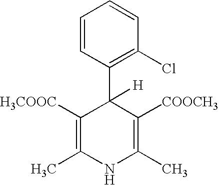
or a salt thereof.

16. Amlodipine or a salt thereof of claim 15, containing less than about 0.15 percent by weight of a compound having a formula: 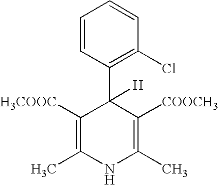
or a salt thereof.
